(19) United States
(12) Patent Application Publication
(10) Pub. No.: US 2016/0361082 A1
(43) Pub. Date: Dec. 15, 2016

(54) SHUT-OFF VALVE FOR THE LIQUID CHANNEL OF A SURGICAL INSTRUMENT

(71) Applicant: OLYMPUS WINTER & IBE GMBH, Hamburg (DE)

(72) Inventors: Pieter BROMMERSMA, Bargteheide (DE); Andreas NOACK, Drage/Schwinde (DE)

(73) Assignee: OLYMPUS WINTER & IBE GMBH, Hamburg (DE)

(21) Appl. No.: 15/114,517

(22) PCT Filed: Jan. 15, 2015
(86) PCT No.: PCT/EP2015/000062

§ 371 Date: Jul. 27, 2016

(30) Foreign Application Priority Data
Feb. 3, 2014 (DE) 10 2014 001 251.4

(51) Int. Cl. A61B 17/32; F16K 5/02; A61M 39/22

(52) U.S. Cl. CPC A61B 17/320016; A61M 39/22; F16K 5/0207; A61M 2039/229; A61B 2217/007

(57) ABSTRACT

A shut-off valve for the liquid channel of a surgical instrument with a plug which is rotatably mounted and includes a cone part located in the front that includes a cone surface and a handle part located in the rear that includes a handle, wherein the liquid channel passes through the cone part, the cone surface and the housing, wherein the cone part and the handle part are mounted displaceably in the direction of the cone axis and non-rotatably against each other and supported by a spring, wherein the handle part is supported to the rear on the housing and the cone part is supported to the front with a flat contact of the cone surface on the housing, characterized in that a radially outwards or inwards projecting rear pin engages in a rear circumferential groove arranged on the housing or the handle part.

The invention relates to a shut-off valve of the kind referred to in the preamble of claim 1.

Shut-off valves for liquid channels of surgical instruments are used, in particular, for urology, such as typically used in urological resectoscopes with continuous irrigation. These comprise an irrigation fluid feed channel and an irrigation fluid return channel. Both are directly equipped with a shut-off valve on the resectoscope. Those shut-off valves can also be used for gas-carrying channels, for example in laparoscopy.

A simply constructed shut-off valve with a conical plug follows from DE 93 07 601 U. The sealing seat of the plug in the housing is secured by a screw with nut. Disadvantageously, a wrench is needed for dismantling, and during the work, individual parts such as the nut or a washer can get lost.

A generic design follows from DE 198 19 814 C1. In this design, screws are dispensed with. Instead, snap-on connections are used.

A shut-off valve for flow media carrying channels of medical endoscopes is also known from DE 101 26 540 A1, wherein a rotation body equipped with a handle comprises a predetermined breaking point, which after breaking open exposes a snap-on device. In this design screws are likewise dispensed with and a snap-on connection is used instead.

Generic shut-off valves are known for example in single-use construction. These are disposed of after use. If they are to be reused, they require continuous conditioning by cleaning and sterilization. Especially for large-surface contact points, as they are given in the generic design by the cone surface, dismantling is advantageous.

In the generic structure, cone part and handle part are rotationally coupled to each other and connected to each other to be longitudinally displaceable, wherein a spring is arranged between the parts. The handle part must be rotatably supported by the rear of the housing. (The terms “front” and “rear” are used to simplify the description, wherein the cone part is located in “front.”) This results in an application of force onto the cone part that presses it sealed against the housing. The liquid channel to be controlled runs through the cone part, so that it can be shut off by its rotation.

In this design the plug comprised of cone part and handle part is attached in the housing by engagement. The engaging is relatively unproblematic. The disengagement for dismantling is confusing and failure-prone and requires the help of special tools.

The problem addressed by the invention is to create a generic shut-off valve, which can be dismantled easily and trouble-free for conditioning purposes.

This problem is solved with the features of the characterizing part of claim 1.

The handle part according to the invention, which must be rotatably supported by the rear of the housing, is supported by a pin in a circumferential groove, which comprises a passage in a circumferential angular position to the rear. In all other angular positions, the support is provided. If, however, the specific location of passage is set, then the pin can pass through to the rear. The whole handle part is released and with this also the cone part. Thus, the dismantling of the plug is greatly simplified. An angle of rotation adjustment of the handle to a certain position is enough to release the plug. No tools are required to do this.

Advantageously according to claim 2, a circumferential groove is provided on the cone part from which the engaging pin can also protrude in a specific circumferential angle position to the rear. This front groove can be used for different purposes than the rear groove, for example for restricting the angle of rotation.

Advantageously according to claim 3, a connection with a longitudinal groove is provided between the passages of the two circumferential grooves. In this way, the pin of the cone part can exit the housing in the same way as the pin of the handle part.

Preferably according to claim 4, a locking device is provided in one of the circumferential grooves that is located in front of the passage and prevents accidental unlocking. Advantageously according to claim 5, the locking device is arranged on the cone part and can be unlocked by moving the cone part in the direction of the axis of rotation.

Advantageously according to claim 6, an inclined infeed is provided on the groove of the handle part. When assembling and also during reassembly of the shut-off valve, the handle part must be pushed in forward direction against the force of the spring until the associated pin can be rotated into the rear groove. The inclined infeed makes this process easier.

The grooves necessary for control of the plug in the inner circumferential surface of the housing must be worked in from the inside in this circumferential surface, which is relatively complex. Advantageously according to claim 7, the inside wall of the housing is therefore lined with a socket from which the grooves are cut. The socket can be made separately from the housing and can be bonded or welded after cutting out the grooves in the housing.

In the drawing, the invention is shown by way of example and schematically.

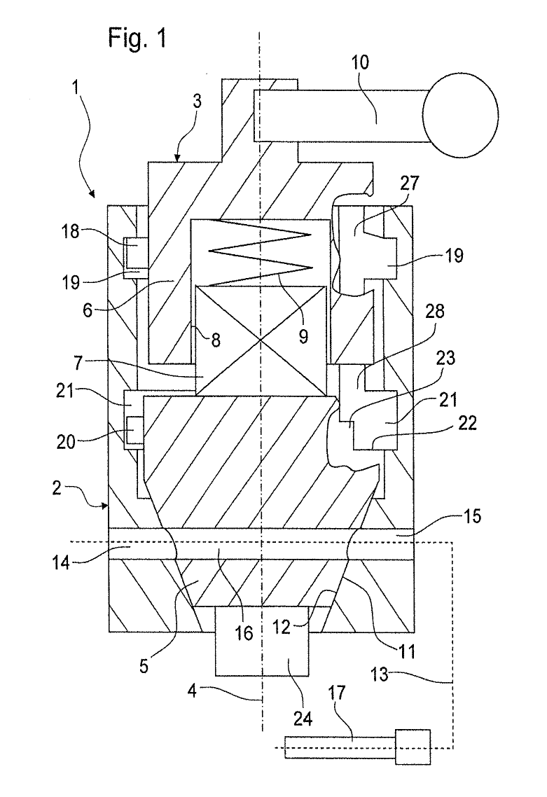

FIG. 1 shows an axial section through a shut-off valve according to the invention with the housing and plug and

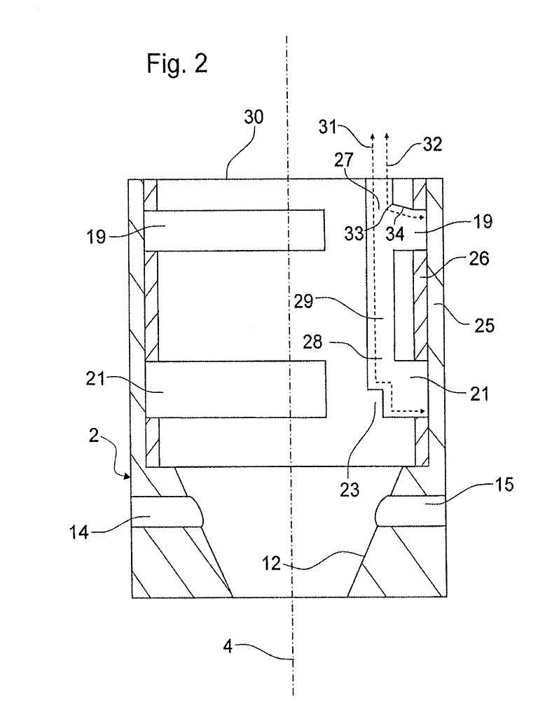

FIG. 2 shows the representation of FIG. 1 without the plug.

FIG. 1 shows a shut-off valve 1, which comprises a housing 2, which is also represented in FIG. 2. In the housing 2 a plug 3 is arranged rotatably about an axis of rotation 4, which is represented only in FIG. 1.

The plug 3 in turn comprises two parts, namely a cone part 5 and a handle part 6, both of which are arranged concentrically to the axis of rotation 4 and are arranged to be movable towards each other in their direction. In addition, the cone part 5 with a square 7 slides in a square bore 8 of the handle part 6. Square 7 and square bore 8 form a sliding guide 7, 8, with which the two parts 5 and 6 are mounted longitudinally displaceable next to each other with simultaneous loosening protection. Between the two parts 5 and 6, a spring 9 is arranged that presses apart the two parts 5 and 6 in the direction of the axis of rotation 4.

A handle 10 is attached on the handle part 6, and makes it possible to rotate the handle part 6, which takes the cone part 5 along via the sliding guide 7, 8.

The two parts 5 and 6 forming plug 3 are mounted rotatably around the axis of rotation 4 in the interior of the housing 2. Thereby, the cone part 5 with its cone surface 11 sits rotatably and surface sealed in a cone seat 12 formed inside the housing 2. The handle part 6 is arranged without guide on the housing 2 and is essentially guided via the sliding guide 7, 8 on the cone part 5.

A liquid channel 13 passes with two parts 14 and 15 through the housing 2 and with a part 16 through the cone part 5. As an exemplary embodiment shown in FIG. 1, the liquid channel 13 supplies a highly schematic illustrated endoscope 17.

The shut-off valve 1 can for example be arranged as in conventional construction directly on the endoscope.

If the handle 10 is actuated, the handle part 6 rotates and in its turning movement takes the cone part 5 along. In doing so, the middle part 16 of the liquid channel 13 is turned and interrupts the liquid channel 13. If it is turned back again, the passage can again be restored.

To improve clarity, the terms “front” and “rear” are used in the present description. These relate to the direction of the axis of rotation 4. On this, “front” is where the cone part 5 is located and “rear” is where the handle part 6 or the handle 10 is located. Thus, the handle part 6 is located to the “rear” of cone part 5.

The handle part 6 is in its circumference designed in cylindrical shape and carries there a radially projecting rear pin 18. This runs in a rear circumferential groove 19, which is made in a cylindrical inner surface of the housing 2.

In a corresponding manner, a front pin 20 is attached on the cylindrical part of the outer surface of the cone part 5 and runs in a front circumferential groove 21 of the housing 2.

As FIG. 1 reveals, the pins 18, 20 and the circumferential grooves 19, 21 serve different purposes. The spring 9 between the cone part 5 and the handle part 6 presses apart these two parts in the direction of the axis of rotation 4. Thereby, the cone part 5 with its cone surface 11 is pressed against the cone seat 12. Thereby, the handle part 6 at the rear part of the plug 3 has to be supported against the housing 2. The set-up of the rear pin 18 against the rear edge of the rear circumferential groove 19, as shown in FIG. 1, takes care of that.

The front pin 20 is not needed for the described generation of the pressing force in plug 3. This is used for limiting an angle of rotation. As FIG. 1 shows, the front edge 22 of the front circumferential groove 21 is provided with a locking device in form of step 23 that is elevating toward the rear.

Upon rotation of the cone part 5, the front pin 20 comes up against step 21 and blocks the further rotation.

The thus-created locking device is unlockable. Thereto, the cone part 5 as a whole is pressed to the rear. The pressure with a finger on an axis lug 24 on the front end surface of the cone part 5 allows this. The cone part 5 can thereby be moved in direction toward the rear. This shift is resiliently absorbed in the sliding guide 7, 8. It allows the front pin 20 located for this purpose in the slightly wider front circumferential groove to overcome the step 23, and thus the cone part 5 can rotate further.

The illustrated shut-off valve 1 is intended for use in surgical instruments and, therefore, must be conditioned before each use, thus cleaned and sterilized. The biggest problem is the large engagement surface at the cone part 5, namely between the cone surface 11 and the seat 12 of the housing 2. These preferably very close-fitting surfaces are best cleaned in the disassembled state.

An endoscope 17 with an attached shut-off valve 1 is frequently conditioned. Therefore, the dismantling of the plug 3 has to be very easily possible. The design shown allows this, as explained below.

FIG. 2 shows the housing 2, which completely coincides with the illustration in FIG. 1 except for one deviation. In the embodiment of FIG. 1, the grooves 19 and 21 have been prepared in the wall of the housing 2, for example by milling out from the inside. Millings from the inside are complicated and expensive. Therefore, the embodiment variant of FIG. 2 is advantageous, in which the inner part of the peripheral wall 25 is formed as an inserted sleeve 26. In the sleeve 26, the grooves are provided as cutouts. The sleeve 26 can be fixated for example by gluing or welding. The design of the housing shown in FIG. 2 is functionally identical to that of FIG. 1. The manufacturing, however, is simpler and less expensive.

The sleeve 26 can be comprised of a plurality of parts separated by the grooves 19 and 21. However, they have to be accurately aligned to each other when mounted on the housing 2, which is complex. Therefore, as shown in FIG. 2, a one-piece sleeve 26 is provided, in which despite the groove cuts all parts are connected. The sleeve can therefore be mounted in precise prefabricated fit in very simple manner.

The guide through the grooves 19, 21 is easier to see in FIG. 2 because there the whole plug 3 is omitted.

In FIG. 2, in turn, the two circumferential grooves 19 and 21 are recognizable. They comprise a passage in the same circumferential position in the respective rear edge. The rear circumferential groove 19 here comprises the rear passage 27 and the front circumferential groove 21 comprises the front passage 28.

At this point, the two pins 18, 20 can leave their respective grooves in the direction to the rear.

Between the passages 27 and 28 runs a longitudinal groove 29 that connects the passages 27 and 28. From the rear passage 27, this longitudinal groove 29 extends further to the rear edge 30 of the housing 2.

In the mounted usage position shown in FIG. 1, the pins 18 and 20 sit in their respective grooves 19 and 21. The rear pin 18 is supported in a direction to the rear against the housing 2. The spring 9 presses the cone part 5 into the seat 12. By turning the handle 10, as described, the liquid channel 13 can be opened or closed as shown in FIG. 1.

If the handle 10 is swiveled counterclockwise from the position of FIG. 1 to a little more than 1800, the front pin 20 arrives on the track 31 shown as dashed lines, illustrated in FIG. 2, until it stops at the step 23. If the plug 3 is to be dismantled, the axis lug 24 has to be pressed in the direction to the rear. The front pin 20 can now follow the track 31 shown as dashed lines around two corners, thereby overcoming the step 23 and be pushed into the longitudinal groove 29.

Since, as FIG. 1 shows, the pins 18 and 20 are arranged in the same angular position, during the movement of the front pin 20 on the track 31 shown as dashed lines, the rear pin 18 runs on the track 32 shown as dashed lines and also runs in the longitudinal groove 29. Now, both pins 18 and 20 can be pushed through the longitudinal groove 29 of the housing 2 completely to the rear beyond the edge 30. Thereby, the cone part 5 and the handle part 6, thus the whole plug 3, are pushed out of the housing 2.

After the conditioning, the plug 3 must be mounted again in the housing 2. To do so, the work is carried out in reverse order of disassembly. The plug 3 is therefore set in an angular position in which the two pins 18 and 20 are in the circumferential angular position of the longitudinal groove 29. Now the plug 3 is pushed to the front until the pins 18, 20 are at the respective circumferential grooves 19, 21. By turning of the handle 10, the pins are rotated out of the area of the longitudinal groove 29 and sit only in their respective circumferential grooves 19, 21.

In this assembly step, the spring 9 that sits between the parts 5 and 6 is also tensioned. It presses the handle part 6 in direction to the rear, i.e. against the rear edge of the rear groove 19. This is particularly noticeable when during the subsequent turning of the plug the pin is up against the edge 32 at the intersection of the longitudinal groove 29 with the rear circumferential groove 19. Here, some forces have to be overcome. This problem can be mitigated somewhat if after the corner 33 the rear edge of the rear circumferential groove 19 is provided with an inclined infeed 34, as the figures show.

In the illustrated exemplary embodiment, the pins 18, 20 are attached on the handle part 6 or the cone part 5, while the circumferential grooves 19, 21 are formed on the housing 2. In an alternative embodiment, not shown, the pins 18 or 20 can also be attached to the housing 2 on the handle part 6 and/or the cone part 5 and the circumferential grooves 19 and 21 can be formed on the handle part 6 and the cone part 5. The passages 27 and 28 are to be adapted to that and now need to open in direction to the front. The described reverse arrangement of the pins 18, 20 can, for example, facilitate the manufacturing by simpler assembly of the shut-off valve.

In another alternative embodiment, the locking device designed in form of the step 23 cannot be arranged, as shown, in the front circumferential groove 21, but in the rear circumferential groove 19. In that case the function of the angle of rotation limiting is performed in the rear circumferential groove 19 and the whole guide on the cone part 5, including the front pin 20 and the front circumferential groove 21, can be dispensed with completely, leading to a design simplification.

### LIST OF REFERENCE SIGNS

1 Shut-off valve
2 Housing
3 Plug
4 Axis of rotation
5 Cone part
6 Handle part
7 Square
8 Square bore
9 Spring
10 Handle
11 Cone surface
12 Cone seat
13 Liquid channel
14 Part
15 Part
16 Part
17 Endoscope
18 Rear pin
19 Rear circumferential groove
20 Front pin
21 Front circumferential groove
22 Front edge
23 Step
24 Axis lug
25 Peripheral wall
26 Sleeve
27 Rear passage
28 Front passage
29 Longitudinal groove
30 Rear edge
31 Track
32 Track
33 Corner
34 Inclined infeed

1. A shut-off valve for the liquid channel of a surgical instrument with a plug which is rotatably mounted in a housing and which comprises, in the direction of its axis of rotation one behind the other, a cone part located in the front that comprises a cone surface and a handle part located in the rear that comprises a handle, wherein the fluid channel passes through the cone part the cone surface and the housing, wherein the cone part and the handle part are mounted longitudinally displaceably in the direction of the cone axis and non-rotatably against each other and mutually supported by a spring, wherein the handle part is supported to the rear on the housing and the cone part is supported to the front with a flat contact of the cone surface on the housing, wherein a radially outwards or inwards projecting rear pin, which is arranged on the handle part or on the housing, engages in a rear circumferential groove arranged on the housing or the handle part, which groove in a circumferential angular position comprises a passage to the rear or front.

2. The shut-off valve according to claim 1, wherein a radially outwards or inwards projecting front pin, which is arranged on the cone part or on the housing, engages in a front circumferential groove arranged on the housing or the cone part, which groove in a circumferential angular position comprises a passage to the rear or front.

3. The shut-off valve according to claim 2, wherein a longitudinal groove that extends parallel to the axis of rotation connects the passages of both circumferential grooves.

4. The shut-off valve according to claim 1, wherein in one of the circumferential grooves in direction of rotation in front of the passage an unlockable locking device is provided.

5. The shut-off valve according to claim 4, wherein the locking device is arranged on the cone part and comprises a step that is elevating toward the rear and that the cone part is formed longitudinally displaceable in the direction of the axis of rotation.

6. The shut-off valve according to claim 1, wherein the rear groove in its rear facing edge at the passage comprises an inclined infeed.

7. The shut-off valve according to claim 1, wherein the inner wall of the housing enclosing plug is lined with a socket, out of which the grooves are cut.
